(19) United States
(12) Patent Application Publication
(10) Pub. No.: US 2023/0277609 A1
(43) Pub. Date: Sep. 7, 2023

(54) ANTI-MICROBIAL AND ANTI-INFLAMMATORY COMPOSITION COMPRISING ARTHROSPIRA EXTRACTS AND AN ORGANIC ACID

(71) Applicant: BIOVITE AUSTRALIA PTY LTD, Brighton, Victoria (AU)

(72) Inventors: Leodevico L ILAG, Balwyn (AU); Peter Owen JOHNSTON, Southport (AU); Andres Monroy CEPEDA, Beaumaris (AU)

(73) Assignee: BIOVITE AUSTRALIA PTY LTD, Brighton, Victoria (AU)

(21) Appl. No.: 18/008,167

(22) PCT Filed: Jun. 2, 2021
(86) PCT No.: PCT/AU2021/050549

§ 371 Date: Dec. 3, 2022

(30) Foreign Application Priority Data
Jun. 3, 2020 (AU) 2020901831

(51) Int. Cl. A61K 35/748; A61K 47/12; A61K 47/02; A61K 9/06; A61P 31/02

(52) U.S. Cl. CPC A61K 35/748; A61K 47/12; A61K 47/02; A61K 9/06; A61P 31/02

(57) ABSTRACT

The present disclosure relates to an anti-microbial and/or anti-inflammatory composition comprising an extract of Arthrospira (Spirulina) and at least one organic acid, such as benzoic acid; a process of preparing such a composition; and the use of the same.

### TECHNICAL FIELD

The present disclosure generally relates to a drug composition, in particular to an antimicrobial and/or anti-inflammatory composition, use thereof, and a process of preparing such a composition.

### BACKGROUND ART

Typically, algal extracts have been shown to have multi-functional properties (anti-inflammatory, growth promotion and/or anti-microbial).

However, algal extracts have an unpleasant smell and a blue-green colour which is not appropriate for topical applications. Further, an enzyme present that is responsible for digesting chitin and correlated to anti-fungal activity is sensitive to harsh extraction and decolorizing conditions. Conventionally, phycocyanin is not only responsible for the anti-inflammatory properties of algae but also responsible for the blue-green colour. Thus, de-colorization can lead to loss of anti-inflammatory properties.

Therefore, there is a need for a method of decolorizing and deodorizing microalgae and derivatives thereof, which does not lead to loss of antimicrobial and/or anti-inflammatory activity, and an antimicrobial and/or anti-inflammatory composition prepared from such a method.

### SUMMARY OF INVENTION

The present disclosure is directed to an antimicrobial and/or anti-inflammatory composition and the process for preparing such a composition, which may at least partially overcome at least one of the abovementioned disadvantages or provide the consumer with a useful or commercial choice.

With the foregoing in view, the present disclosure in one form, resides broadly in a process for preparing an antimicrobial and/or anti-inflammatory composition comprising an extract of Arthrospira, and at least one organic acid.

One aspect relates to an antimicrobial and/or anti-inflammatory composition comprising a extract of Arthrospira, and at least one organic acid in an amount of at least about 0.1 wt. % based on the total weight of the composition.

The organic acid may be selected from the group comprising aromatic carboxylic acids, short chain fatty acids, medium and long chain saturated fatty acids, medium and long chain unsaturated fatty acids, polyphenols, phenolic acids, flavonoids, or a combination thereof. The organic acid may be selected from the group comprising benzoic acid, valproic acid, formic acid, acetic acid, propionic acid, butyric acid, isobutyric acid, valeric acid, isovaleric acid, cholorogenic acid, gallic acid, vanillin, salicylic acid, caffeic acid, syringic acid, coumaric acid, ferulic acid, cinnamic acid, quercetin, genstein, kaempferol, lauric acid, palmitic acid, sapienic acid, stearic acid, oleic acid, linoleic acid, gamma linolenic acid, docosodienoic acid, or a combination thereof. For example, the at least one organic acid may be benzoic acid.

In an embodiment, the amount of benzoic acid may be between about 2 wt. % and about 15 wt. % based on the total weight of the dried composition.

In an embodiment, the composition may further comprise titanium dioxide. The amount of titanium dioxide may be between about 0.5 wt. % and about 3.0 wt. % based on the total weight of the composition.

In another aspect there is provided a process for preparing an antimicrobial and/or anti-inflammatory composition comprising an extract of Arthrospira, and at least one organic acid, comprising the following steps:

(i) obtaining an extract of Arthrospira; and

(ii) adding at least one agent to decolorize and/or deodorize the Arthrospira extract to provide the antimicrobial and/or anti-inflammatory composition, wherein the addition of the at least one agent produces the organic acid.

In an embodiment, the agent to decolorize and/or deodorize may be selected from the group comprising hydrogen peroxide, benzoyl peroxide, valproate peroxide, acetyl peroxide, formyl hydroperoxide, peroxypropionic acid, peroxybutyric acid, or a combination thereof. For example, the agent may be selected from hydrogen peroxide and benzoyl peroxide.

In an embodiment, the process further comprises adding at least one organic acid.

In another aspect there is provided a process for preparing an antimicrobial and/or anti-inflammatory composition comprising an extract of Arthrospira, and at least one organic acid, comprising the following steps:

(i) obtaining an extract of Arthrospira; and

(ii) adding at least one organic acid to the Arthrospira extract to provide the antimicrobial and/or anti-inflammatory composition.

In an embodiment, the process further comprises growing and harvesting Arthrospira. In an embodiment, the Arthrospira were/are grown under physiologically stressed conditions. In a alternate embodiment, the Arthrospira were/are not grown under physiologically stressed conditions.

In an embodiment, the process may further comprise the addition of titanium dioxide. The amount of titanium dioxide may be between about 0.5 wt. % and about 3.0 wt. %.

In an embodiment, the at least one organic acid may be selected from the group comprising aromatic carboxylic acids, short chain fatty acids, polyphenols, phenolic acids, flavonoids, or a combination thereof. The at least one organic acid may be selected from the group comprising benzoic acid, valproic acid, valproic acid, formic acid, acetic acid, propionic acid, butyric acid, isobutyric acid, valeric acid, isovaleric acid, cholorogenic acid, gallic acid, vanillin, salicylic acid, caffeic acid, syringic acid, coumaric acid, fuerulic acid, cinnamic acid, quercetin, genstein, or a combination thereof. The at least one organic acid may be benzoic acid. In an embodiment, the amount of organic acid in the composition may be in an amount between about 0.1 wt. % and about 15 wt. % based on the total weight of the composition.

In an embodiment, the amount of the at least one agent to decolorize and/or deodorize may be between about 0.5 wt. % and about 5.0 wt. % based on the total weight of the composition.

In another aspect there is provided a method of treating or preventing a disease or condition responsive to treatment with an Arthrospira extract in a subject, comprising administering to a subject an effective amount of the composition of any one of the embodiments or examples described herein or the composition prepared by the process according to any one of the embodiments or examples described herein.

In another aspect there is provided a use of an extract of Arthrospira in the manufacture of a medicament for treating or preventing a disease or condition responsive to treatment with an Arthrospira extract, wherein the medicament comprises the composition of any one of the embodiments or examples described herein or the composition prepared by the process of any one of the embodiments or examples described herein.

In another aspect there is provided a composition for use in treating or preventing a disease or condition responsive to treatment with an Arthrospira extract, wherein the composition is according to any one of the embodiments or examples described herein or the composition prepared by the process according to any one of the embodiments or examples described herein.

### BRIEF DESCRIPTION OF DRAWINGS

Some embodiments of the present disclosure are described and illustrated herein, by way of example only, with reference to the accompanying Figures in which:

FIG. 1 is a flowchart of a method of producing an antimicrobial and/or anti-inflammatory composition, in accordance with an embodiment of the present invention.

FIG. 2 is a 400 MHZ ¹H NMR (d4-methanol) spectrum of an HPLC fraction of an antimicrobial and/or anti-inflammatory composition prepared according to a process of the present invention.

FIG. 3 is a reverse phase HPLC chromatogram of a methanol extract of an HPLC fraction of an antimicrobial and/or anti-inflammatory composition prepared according to a process of the present invention.

### DESCRIPTION OF EMBODIMENTS

The present disclosure describes the following various non-limiting examples, which relate to investigations undertaken to identify alternative and improved antimicrobial and/or anti-inflammatory composition and processes for preparing the antimicrobial and/or anti-inflammatory composition. The present inventors have prepared an antimicrobial and/or anti-inflammatory composition comprising an extract of Arthrospira, and at least one organic acid. The antimicrobial and/or anti-inflammatory composition can comprise an extract of Arthrospira, and at least one organic acid The present inventors have also identified a process for preparing an antimicrobial and/or anti-inflammatory composition comprising an extract of Arthrospira, and at least one organic acid.

At least according to some embodiments or examples as described herein, the present disclosure provides an alternative or improved antimicrobial and/or anti-inflammatory composition that has been prepared by decolorizing and deodorizing the extract, which improves antimicrobial (for example, chitanase activity), anti-inflammatory and cell growth promoting properties.

### General Terms

Throughout this disclosure, unless specifically stated otherwise or the context requires otherwise, reference to a single step, composition of matter, group of steps or group of compositions of matter shall be taken to encompass one and a plurality (i.e. one or more) of those steps, compositions or matter, groups of steps or groups of composition of matter. Thus, as used herein, the singular forms “a”, “an” and “the” include plural aspects unless the context clearly indicates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

Unless otherwise indicated, the terms “first”, “second”, etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

Reference herein to “example”, “one example”, “another example”, or similar language means that one or more feature, structure, element, component or characteristic described in connection with the example is included in at least one embodiment or implementation. Thus, the phrases “in one example”, “as one example”, and similar language throughout the present disclosure may, but do not necessarily, refer to the same example. Further, the subject matter characterizing any one example may, but does not necessarily, include the subject matter characterizing any other example.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

As used herein, the term “about”, unless stated to the contrary, refers to +/−10%, typically +/−5%, typically +/−1%, of the designated value.

As used herein, the term “subject” refers to any organism that is susceptible to a disease or condition. For example, the subject can be an animal, a mammal, a primate, a livestock animal (e.g., sheep, cow, horse, pig), a companion animal (e.g., dog, cat), or a laboratory animal (e.g., mouse, rabbit, rat, guinea pig, hamster). In one example, the subject is a mammal. In one preferred embodiment, the subject is human. In one alternative embodiment, the subject is a non-human animal.

As used herein, the term “treating” includes alleviation of one or more symptoms associated with a specific disease or condition.

As used herein, the term “prevention” includes prophylaxis of the specific disease or condition.

Herein “weight %” may be abbreviated to as “wt %”.

Antimicrobial and/or Anti-Inflammatory Composition

The present disclosure provides an antimicrobial and/or anti-inflammatory composition comprising an extract of Arthrospira, and at least one organic acid. The antimicrobial and/or anti-inflammatory composition can comprise an extract of Arthrospira, and at least one organic acid in an amount of at least about 0.1 wt. % based on the total weight of the composition.

“Arthrospira” is a biomass of cyanobacteria (blue-green algae) that can be consumed by humans and animals. The Arthrospira may be selected from Arthrospira maxima (A. maxima) and Arthrospira platensis (A. platensis), or a combination thereof.

In some embodiments, the amount of Arthrospira extract in the composition can vary depending on the application. For example, the amount of Arthrospira extract in the composition may be between about 0.01 wt. % and about 99 wt. % based on the total weight of the composition. The Arthrospira extract can be provided in an amount (as a weight % based on total weight of the composition) of less than about 99, 95, 90, 85, 80, 75, 70, 65, 60, 55, 50, 45, 40, 35, 30, 25, 20, 15, 10, 5, 1, 0.5, or 0.1. The Arthrospira extract can be provided in an amount (as a weight % based on total weight of the composition) of at least about 0.1, 0.5, 1, 5, 10, 15, 20, 25, 30, 35, 40, 45, 50, 55, 60, 65, 70, 75, 80, 85, 90, 95 or 99. The Arthrospira extract can be in a range provided by any two of these upper and/or lower amounts, for example between about 50 to about 99% wt. % of Arthrospira extract present in the composition.

It is known that a number of organic acids (e.g. benzoic acid) may be found naturally in the extract of Arthrospira. However, the inventors have surprisingly found that organic acid is also generated as a by-product during the process for preparing the antimicrobial and/or anti-inflammatory composition, as described herein. This unexpected finding may enhance the antimicrobial and/or anti-inflammatory properties of the composition. Further advantages may also be provided by the addition of one or more organic acids.

In some embodiments, the organic acid may be selected from the group comprising aromatic carboxylic acids, short chain fatty acids, medium and long chain saturated fatty acids, medium and long chain unsaturated fatty acids, polyphenols, phenolic acids, flavonoids, or a combination thereof. The organic acid may be selected from the group comprising benzoic acid, valproic acid, formic acid, acetic acid, propionic acid, butyric acid, isobutyric acid, valeric acid, isovaleric acid, cholorogenic acid, gallic acid, vanillin, salicylic acid, caffeic acid, syringic acid, coumaric acid, ferulic acid, cinnamic acid, quercetin, genstein, kaempferol, lauric acid, palmitic acid, sapienic acid, stearic acid, oleic acid, linoleic acid, gamma linolenic acid, docosodienoic acid, or a combination thereof. In one example, the organic acid may be benzoic acid, valproic acid, or a combination thereof. For example, the organic acid may be benzoic acid.

In one embodiment, the amount of organic acid in the composition may be between about 0.1 wt. % and about 15.0 wt. % based on the total weight of the composition. The organic acid can be provided in an amount (as a weight % based on total weight of the composition) of less than about 15, 14, 13, 12, 11, 10, 9.8, 9.6, 9.4, 9.2, 9.0, 8.8, 8.6, 8.4, 8.2, 8.0, 7.8, 7.6, 7.4, 7.2, 7.0, 6.8, 6.6, 6.4, 6.2, 6.0, 5.8, 5.6, 5.4, 5.2, 5.0, 4.8, 4.6, 4.4, 4.2, 4.0, 3.8, 3.6, 3.4, 3.2, 3.0, 2.8, 2.6, 2.4, 2.2, 2.0, 1.8, 1.6, 1.4, 1.2, 1.0, 0.8, 0.6, 0.4, 0.2 or 0.1. The organic acid can be provided in an amount (as a weight % based on total weight of the composition) of at least about 0.1, 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10, 11, 12, 13, 14 or 15. The organic acid can be in a range provided by any two of these upper and/or lower amounts, for example between about 2.0 to about 10.0 wt % of the total weight of the composition. In one example, the amount of benzoic acid may be between about 0.1 wt. % and about 15 wt. % based on the total weight of the composition. For example, the amount of benzoic acid may be between about 2 wt. % and about 15 wt. % based on the total weight of the dried composition.

In an embodiment, the composition may further comprise titanium dioxide. For example, the amount of titanium dioxide present in the composition may be in an amount between about 0.5 wt. % and about 3.0 wt. % based on the total weight of the composition. The titanium dioxide can be provided in an amount (as a weight % based on total weight of the composition) of less than about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6 or 0.5. The titanium dioxide can be provided in an amount (as a weight % based on total weight of the composition) of at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0. The titanium dioxide can be in a range provided by any two of these upper and/or lower amounts.

In another embodiment, the composition may further comprise a diluent, wherein the diluent may be water. The composition may comprise a small residual amount of water (i.e. a moisture content). In some embodiments, the composition has a moisture content of less than about 20, 19, 18, 17, 16, 15, 14, 13, 12, 11, 10, 9, 8, 7, 6, 5, 5.5, 4, 4.5, 3, 2.5, 2, 1.5, 1.4, 1.3, 1.2, 1.1, 1, 0.9, 0.8, 0.7, 0.6 or 0.5 wt. % water, typically less than 10 wt. %, preferably less than 5 wt. %, e.g. 3 wt. % or less. Combinations of these values can provide a range selection, for example between about 3 to about 5 wt. % water.

In another embodiment, the composition may be formulated for administration to a mammal by topical administration. The composition may be in the form of a powder, emulsion, cream or lotion.

In one embodiment, an antimicrobial and/or anti-inflammatory composition may comprise:
(i) an extract of Arthrospira in an amount of about 50 wt. % to about 99 wt. % based on the total weight of the composition;
(ii) titanium dioxide in an amount of about 0.5 wt. % to about 3.0 wt. % based on the total weight of the composition; and
(iii) optionally at least one organic acid in an amount of about 0.1 wt. % to about 15 wt. % based on the total weight of the composition.

An exemplified composition may comprise:
(i) an extract of Arthrospira in an amount of about 50 wt. % to about 99 wt. % based on the total weight of the composition;
(ii) titanium dioxide in an amount of about 0.5 wt. % to about 3.0 wt. % based on the total weight of the composition; and
(iii) at least one organic acid in an amount of about 0.1 wt. % to about 15 wt. % based on the total weight of the composition.

An exemplified dried composition may comprise:
(i) an extract of Arthrospira in an amount of about 50 wt. % to about 99 wt. % based on the total weight of the dried composition;
(ii) titanium dioxide in an amount of about 0.5 wt. % to about 3.0 wt. % based on the total weight of the dried composition; and
(iii) at least one organic acid in an amount of about 2 wt. % to about 15 wt. % based on the total weight of the dried composition.

The inventors have identified from a composition analysis of an antimicrobial and/or anti-inflammatory composition as described herein unexpectedly shows that other flavonoids, polyphenols/phenolic acids and fatty acids present in significant quantities when compared with prior art compositions, which may provide further advantages. For example, flavonoids, phenolics and fatty acids are a class of compounds with known anti-microbial and anti-inflammatory properties which may further enhance the antimicrobial and/or anti-inflammatory properties of the composition.

Flavanoids and phenolics are known to be present in microalgae such as Arthrospira. It has been reported that depending on growth conditions, a species of Arthorspira maxima contains 0.45-1.7 grams of phenolics per 100 grams dried weight of algae and 0.135-0.500 grams of phenolics per 100 grams dried weight of algae (REF: HHA El-Baky, FK El-Baz, GS El-Baroty (2009) “Production of phenolic compounds from Spirulina maxima microalgae and its protective effects.” African Journal of Biotechnology Vol. 8 (24), pp. 7059-7067.

Polyphenols (compounds with two or more phenol groups) are a class of phytochemicals found in a variety of botanical sources. Natural polyphenols can range from simple molecules, such as phenolic acids to large highly polymer sized compounds such as tannins. Polyphenols (or phenolics) all have a common basic chemical component, a phenolic ring structure. Polyphenols can exist in their free form, or as polyphenol glycosides. Conjugated forms of polyphenols are the most common, where various sugar molecules, organic acids and lipids (fats) are linked with the phenolic ring structure. Despite having a common phenolic ring structure, differences in the conjugated chemical structure and size. Phenolic acids are simple molecules such as caffeic acid, vanillin, and coumaric acid. Phenolic acids form a diverse group that includes the widely distributed hydroxybenzoic and hydroxycinnamic acids (despite the latter two only having one phenolic ring). Hydroxycinnamic acid compounds (p-coumaric, caffeic acid, ferulic acid) occur most frequently as simple esters with hydroxy carboxylic acids or glucose, while the hydroxybenzoic acid compounds (p-hydroxybenzoic, gallic acid, ellagic acid) are present mainly in the form of glucosides. Flavonoids are a class of polyphenols that are secondary metabolites from plants and often consumed. The basic structure of flavonoids is a 15-carbon skeleton, which consists of two phenyl rings and a heterocyclic ring containing an embedded oxygen. Examples of flavonoids include catechins, anthocyanidins, quercetin, kaempferol and genistein.

Fatty acids are also known to be present in microalgae such as Arthrospira. Fatty acids are carboxylic acids with an aliphatic chain, which is either saturated or unsaturated. The aliphatic chain of short chain fatty acids range from 1-5 carbon atoms. Medium chain fatty acids have 6-12 carbon atoms comprising the aliphatic chains, while long chain fatty acids range from greater than 13 carbon atoms. Fatty acids are major components of cellular membranes in most biological systems including microalgae.

In an embodiment, the composition may further comprise flavonoids. For example, the amount of flavanoids present in the composition may be in an amount between about 0.5 g/100 g and about 10.0 g/100 g based on the total weight of the composition. The flavonoids can be provided in an amount (as g/100 g or % based on total weight of the composition) of less than about 10, 9.5, 8, 8.5, 8, 7.5, 7, 6.5, 6, 5.5, 5, 4.5, 4, 3.5, 3, 2.5, 2, 1.5, 1 or 0.5. The flavonoids can be provided in an amount (as g/100 g or % based on total weight of the composition) of at least about 0.5, 1, 1.5, 2, 2.5, 3, 3.5, 4, 4.5, 5, 5.5, 6, 6.5, 7, 7.5, 8, 8.5, 9, 9.5 or 10. Combinations of these values can provide a range selection, for example between about 1 to about 5 g/100 g. In a particular example, the amount of flavanoids present in the composition may be about 2.5 g/100 g.

In an embodiment, the composition may further comprise phenolic acids/polyphenols. For example, the amount of phenolic acids/polyphenols present in the composition may be in an amount between about 0.1 g/100 g and about 5.0 g/100 g based on the total weight of the composition. The phenolic acids/polyphenols can be provided in an amount (as g/100 g or % based on total weight of the composition) of less than about 5, 4.5, 4, 3.5, 3, 2.5, 2, 1.5, 1, 0.8, 0.6, 0.5, 0.4, 0.3, 0.2 or 0.1. The phenolic acids/polyphenols can be provided in an amount (as g/100 g or % based on total weight of the composition) of at least about 0.1, 0.2, 0.3, 0.4, 0.5, 0.6, 0.8, 1, 1.5, 2, 2.5, 3, 3.5, 4, 4.5 or 5. Combinations of these values can provide a range selection, for example between about 0.2 to about 2 g/100 g. In a particular example, the amount of phenolic acids/polyphenols present in the composition may be about 0.6 g/100 g.

In an embodiment, the composition may further comprise fatty acids. For example, the amount of total fatty acids (saturated and unsaturated) present in the composition may be in an amount between about 0.5 g/100 g and about 10.0 g/100 g based on the total weight of the composition. The total fatty acids can be provided in an amount (as g/100 g or % based on total weight of the composition) of less than about 10, 9.5, 8, 8.5, 8, 7.5, 7, 6.5, 6, 5.5, 5, 4.5, 4, 3.5, 3, 2.5, 2, 1.5, 1 or 0.5. The total fatty acids can be provided in an amount (as g/100 g or % based on total weight of the composition) of at least about 0.5, 1, 1.5, 2, 2.5, 3, 3.5, 4, 4.5, 5, 5.5, 6, 6.5, 7, 7.5, 8, 8.5, 9, 9.5 or 10. Combinations of these values can provide a range selection, for example between about 2 to about 6 g/100 g. In a particular example, the amount of total fatty acids present in the composition may be about 4 g/100 g.

Method for Preparing an Antimicrobial and/or Anti-Inflammatory Composition

Disclosed herein is a method of preparing an antimicrobial and/or anti-inflammatory composition described herein.

In one embodiment, there is provided a process for preparing an antimicrobial and/or anti-inflammatory composition comprising an extract of Arthrospira, and at least one organic acid, comprising the following steps: (i) obtaining an extract of Arthrospira; and (ii) adding at least one agent to decolorize and/or deodorize the Arthrospira extract to provide the antimicrobial and/or anti-inflammatory composition, wherein the addition of the at least one agent produces the organic acid.

The process may further comprise adding at least one organic acid.

In another embodiment, there is provided a process for preparing an antimicrobial and/or anti-inflammatory composition comprising an extract of Arthrospira, and at least one organic acid, comprising the following steps: (i) obtaining an extract of Arthrospira; and (ii) adding at least one organic acid to the Arthrospira extract to provide the antimicrobial and/or anti-inflammatory composition.

The process comprises a plurality of phases. The plurality of phases includes a first phase of growing the Arthrospira, harvesting the Arthrospira, optionally stressing the Arthrospira, and washing and drying the Arthrospira. In a particularly preferred embodiment, the Arthrospira is A. maxima. At the end of this first phase, the resultant product is a fine, uniform dark green powder, known as the “biosource”. It will be appreciated that the term “biosource” is also referred to as “extract of Arthrospira” and also corresponds to step (i) of the process of obtaining the extract of the Arthrospira.

In some embodiments, the process may further comprise growing and harvesting Arthrospira. The Arthrospira may be grown under physiologically stressed conditions. The biosource or extract of Arthrospira can comprise intact Arthrospira filaments, segments of the filaments, disrupted/lysed segments or extracts/fractions thereof. The extract of Arthrospira can comprise Arthrospira that is alive or that is not alive. Preferably, the extract of Arthrospira comprises disrupted Arthrospira filaments and segments. The extract of Arthrospira can be prepared in any suitable way, provided that the biocidal activity is not compromised. Normally, this would involve the steps of: (I) growing Arthrospira in any suitable way; (II) harvesting the grown Arthrospira in any suitable way; and (III) drying the Arthrospira in any suitable way.

Optional steps include: additionally physiologically stressing the Arthrospira in any suitable way; disrupting the filaments and segments in any suitable way, sterilising the organism in any suitable way, removing or degrading the chlorophyll in any suitable way, and milling the dried Arthrospira. Some of the above steps are described, for example, in the specifications of New Zealand Patents No. 336620 and 336619, and U.S. Pat. No. 9,498,504 and WO2006047830, the entire contents of which are incorporated herein by cross reference. For example, stressing can be carried out at the time of harvest by either depriving the Arthrospira of essential nutrients (nutrient diminution) or light for a period sufficient to stress it but not kill it by management of the growing conditions. Stressing can also be by partial desiccation or by harvesting the Arthrospira and keeping it alive in damp conditions until stressed but not dead.

The process may further comprise rinsing and/or drying the Arthrospira extract to provide a dried Arthrospira powder. It will be appreciated that the extract of Arthrospira is typically prepared by harvesting Arthrospira, washing the harvested Arthrospira, and drying the washed Arthrospira. Some suppliers mill the Arthrospira whilst wet. Some of the above steps are described, for example, in the specifications of U.S. Pat. No. 9,498,504 and WO2006047830, the entire contents of which are incorporated herein by cross reference. For example, Arthrospira can be spray dried to form a fine powder of disrupted cells. Spray drying may take place, for example, at 50-190° C. for a few seconds. For example, drying can be carried out in any suitable way, including by cyclonic drying, heat pump drying, heat tube drying, refractory drying or thermal drying under about 67° C. for short periods of time. For example, filaments can be disrupted using milling or explosive decompression as described in New Zealand Patents No. 328013 and No. 328740, the entire contents of which are incorporated herein by cross reference.

The plurality of phases includes a second phase of processing to provide a “wet pre-mix intermediate” and quality control testing of that “wet pre-mix intermediate”. The second phase in the process to prepare the “wet pre-mix intermediate” may correspond to step (ii) adding at least one agent to decolorize and/or deodorize the Arthrospira extract to provide the antimicrobial and/or anti-inflammatory composition, wherein the addition of the at least one agent produces the organic acid. In an alternative embodiment, the second phase may also correspond to step adding at least one organic acid to the Arthrospira extract to provide the antimicrobial and/or anti-inflammatory composition.

The plurality of phases includes an optional third phase of drying the “wet pre-mix intermediate”, and milling the resultant dried material to produce a powder, which is the antimicrobial and/or anti-inflammatory composition.

In one embodiment, the agent to decolorize and/or deodorize may be selected from the group comprising hydrogen peroxide, benzoyl peroxide, valproate peroxide, acetyl peroxide, formyl hydroperoxide, peroxypropionic acid, peroxybutyric acid, or a combination thereof. In an example, the agent may be selected from hydrogen peroxide, benzoyl peroxide, or a combination thereof. For example, the agent may be hydrogen peroxide and benzoyl peroxide.

In one embodiment, the organic acid may be produced as a by-product of decolorizing and/or deodorizing the “biosource” during production of the “wet pre-mix intermediate”, and has at least one of anti-fungal activity, antimicrobial activity, and a combination thereof.

In another embodiment, the organic acid may be added during formulation of the “wet pre-mix intermediate” to form the antimicrobial and/or anti-inflammatory composition.

In yet another embodiment, the organic acid may be added to the dried antimicrobial and/or anti-inflammatory composition.

The process may further comprise addition of titanium dioxide. The inventors have found that addition of titanium dioxide to the composition acts as a catalyst for breakdown of residual hydrogen peroxide and also has a colour lightening effect.

The at least one organic acid may be selected from the group comprising aromatic carboxylic acids, short chain fatty acids, medium and long chain saturated fatty acids, medium and long chain unsaturated fatty acids, polyphenols, phenolic acids, flavonoids, or a combination thereof. The at least one organic acid may selected from the group comprising benzoic acid, valproic acid, valproic acid, formic acid, acetic acid, propionic acid, butyric acid, isobutyric acid, valeric acid, isovaleric acid, cholorogenic acid, gallic acid, vanillin, salicylic acid, caffeic acid, syringic acid, coumaric acid, ferulic acid, cinnamic acid, quercetin, genstein, kaempferol, lauric acid, palmitic acid, sapienic acid, stearic acid, oleic acid, linoleic acid, gamma linolenic acid, docosodienoic acid, or a combination thereof. For example, the organic acid may be benzoic acid, valproic acid, or a combination thereof. In a particular example, the organic acid may be benzoic acid.

The process may further comprise drying the composition to provide a dried powder composition.

In one embodiment, the amount of organic acid may be between about 0.1 wt. % and about 15.0 wt. % based on the total weight of the composition. The organic acid can be provided in an amount (as a weight % based on total weight of the composition) of less than about 15, 14, 13, 12, 12, 10, 9.8, 9.6, 9.4, 9.2, 9.0, 8.8, 8.6, 8.4, 8.2, 8.0, 7.8, 7.6, 7.4, 7.2, 7.0, 6.8, 6.6, 6.4, 6.2, 6.0, 5.8, 5.6, 5.4, 5.2, 5.0, 4.8, 4.6, 4.4, 4.2, 4.0, 3.8, 3.6, 3.4, 3.2, 3.0, 2.8, 2.6, 2.4, 2.2, 2.0, 1.8, 1.6, 1.4, 1.2, 1.0, 0.8, 0.6, 0.4, 0.2 or 0.1. The organic acid can be provided in an amount (as a weight % based on total weight of the composition) of at least about 0.1, 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10, 11, 12, 13, 14, or 15. The organic acid can be in a range provided by any two of these upper and/or lower amounts, for example between about 2 to about 15 wt. % of the total weight of the composition. In one example, the amount of benzoic acid may be between about 2 wt. % and about 15 wt. % based on the total weight of the dried composition.

In an embodiment, the process may further comprise addition of titanium dioxide. The amount of titanium dioxide present in the composition may be between about 0.5 wt. % and about 3.0 wt. % based on the total weight of the composition. The titanium dioxide can be provided in an amount (as a weight % based on total weight of the composition) of less than about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6 or 0.5. The titanium dioxide can be provided in an amount (as a weight % based on total weight of the composition) of at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0. The titanium dioxide can be in a range provided by any two of these upper and/or lower amounts.

In another embodiment, the process may further comprise addition of a diluent, wherein the diluent may be water. During the process the composition may comprise a small residual amount of water (i.e. a moisture content). In some embodiments, the composition has a moisture content of less than about 20, 19, 18, 17, 16, 15, 14, 13, 12, 11, 10, 9, 8, 7, 6, 5, 5.5, 4, 4.5, 3, 2.5, 2, 1.5, 1.4, 1.3, 1.2, 1.1, 1, 0.9, 0.8, 0.7, 0.6 or 0.5 wt. % water, typically less than 10 wt. %, preferably less than 5 wt. %, e.g. 3 wt. % or less. Combinations of these values can provide a range selection, for example between about 3 to about 5 wt. % water.

In an embodiment, the process addition of an agent to decolorize and/or deodorize in an amount between about 0.5 wt. % and about 5.0 wt. % based on the total weight of the composition. For example, the amount of agent may be added in an amount between about 0.5 wt. % and about 5.0 wt. % based on the total weight of the composition. The agent can be provided in an amount (as a weight % based on total weight of the composition) of less than about 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6 or 0.5. The agent can be provided in an amount (as a weight % based on total weight of the composition) of at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0. The agent can be in a range provided by any two of these upper and/or lower amounts.

Once an antimicrobial and/or anti-inflammatory composition is obtained, it will be introduced into an appropriate storage vessel. For example, a vessel that allows the antimicrobial and/or anti-inflammatory composition dried to be delivered in a predetermined manner such as topically.

In one embodiment, an antimicrobial and/or anti-inflammatory composition may be obtained from a process comprising:

optionally growing and harvesting Arthrospira, and optionally stressing the Arthrospira, to obtain an extract of Arthrospira;
(ii) optionally drying the extract of Arthrospira;
(iii) optionally adding water while stirring;
(iv) adding at least one agent to decolorize and/or deodorize the Arthrospira extract to the mixture of step (ii) while stirring and mixing continues;
(v) optionally adding titanium dioxide to the mixture of step (iii) while stirring mixing continues;
(vi) mixing the resulting mixture of (i) to (iv) for a period to obtain a wet pre-mix intermediate;
(vii) optionally drying the wet pre-mix intermediate;
(viii) obtaining an antimicrobial and/or anti-inflammatory composition;
(ix) optionally adding organic acid to the final composition to an amount of up to about 15 wt. % based on the total weight of the composition; add
(x) optionally drying the final composition.

A person skilled in the art would appreciate that steps (i) to (v) can be undertaken in any order.

The period of time in step (v) may be: at least about 30 hours; at least about 35 hours; at least about 40 hours; at least about 45 hours; at least about 50 hours; at least about 55 hours; at least about 60 hours; at least about 65 hours; at least about 70 hours; at least about 75 hours; at least about 80 hours; at least about 85 hours; at least about 90 hours; at least about 95 hours; or at least about 100 hours.

Following or before the addition of the first component into the vessel a stirring means, such as a stirring bar or internal stirrer, may be engaged and used sporadically or continuously throughout the manufacture of the antimicrobial and/or anti-inflammatory composition.

In one embodiment, the temperature of the mixture does not exceed 40° C. For example, the temperature of the mixture can be maintained at less than about 40° C.

For example, drying can be carried out in any suitable way, including by cyclonic drying, heat pump drying, heat tube drying, refractory drying or thermal drying under about 67° C. for short periods of time.

An exemplified process comprises the following steps:
(i) obtaining an extract of Arthrospira;
(ii) drying the extract of Arthrospira;
(iii) adding water while stirring;
(iv) adding at least one agent to decolorize and/or deodorize the Arthrospira extract to the mixture of step (ii) while stirring and mixing continues;
(v) adding titanium dioxide to the mixture of step (iii) while stirring mixing continues;
(vi) allowing at least 48 hours of mixing to provide a wet pre-mix intermediate; and
(vii) adding organic acid to the final composition to an amount of up to about 15 wt. % based on the total weight of the composition.

Steps (i) to (v) may be undertaken in any order.

It will be appreciated that an agent to decolorize and/or deodorize the Arthrospira extract may be present in the final composition in residual amounts. The agent may i) be converted to an organic acid by a chemical reaction that occurs during the composition preparation process and/or ii) act as a catalyst. The agent may be selected from the group comprising hydrogen peroxide, benzoyl peroxide, valproate peroxide, acetyl peroxide, formyl hydroperoxide, peroxypropionic acid, peroxybutyric acid, or a combination thereof. For example, the agent may be selected from hydrogen peroxide, benzoyl peroxide, or a combination thereof. In one example, the agent may be hydrogen peroxide and benzoyl peroxide.

The composition may comprise a small residual amount of agent. In some embodiments, the composition may comprise less than about 0.06, 0.04, 0.02, 0.01, 0.008, 0.006, 0.004, 0.002 or 0.001% v/w of the agent, typically less than 0.04% v/w, e.g. 0.02% v/w or less. Combinations of these values can provide a range selection, for example between about 0.002 to about 0.02% v/w of the agent present in the composition.

Antimicrobial and/or Anti-Inflammatory Formulation

Formulations suitable for use in the methods and uses described herein comprise a formulation comprising an antimicrobial and/or anti-inflammatory composition, i.e. a composition comprising an extract of Arthrospira, and at least one organic acid in an amount of at least about 0.1 wt. % based on the total weight of the composition, as described herein. In some embodiments, the formulation an extract of Arthrospira, and at least one organic acid is presented as a pharmaceutical and/or nutraceutical formulation. In some embodiments, there is provided the use or method as described herein, wherein the formulation comprising an extract of Arthrospira, and at least one organic acid, is administered in the form of a pharmaceutical and/or nutraceutical formulation.

The formulations described herein may contain a formulation comprising an antimicrobial and/or anti-inflammatory composition comprising an extract of Arthrospira, and at least one organic acid. The antimicrobial and/or anti-inflammatory composition, by weight %, may be present in the formulation in an amount of less than about 99, 95, 90, 85, 80, 75, 70, 65, 60, 55, 50, 45, 40, 35, 30, 25, 20, 15, 10, 5, 1, 0.5, or 0.1. The antimicrobial and/or anti-inflammatory composition, by weight %, may be present in the formulation in an amount of at least about 0.1, 0.5, 1, 5, 10, 15, 20, 25, 30, 35, 40, 45, 50, 55, 60, 65, 70, 75, 80, 85, 90, 95 or 99. The antimicrobial and/or anti-inflammatory composition can be present in a range provided by any two of these upper and/or lower amounts, for example between about 4 to about 30 wt. %.

In some embodiments, the formulation comprises or consists of an antimicrobial and/or anti-inflammatory composition comprising an extract of Arthrospira, and at least one organic acid, optionally a carrier, and optionally one or more additives.

### Carrier

The formulation may, for example, comprise a liquid carrier. The liquid carrier may be an aqueous liquid carrier. The liquid carrier may be selected from the group comprising water, paraffin, medium-chain triglyceride (MCT) oil, olive oil, rice bran oil, soya oil, coconut oil, palm oil, grape seed oil, vegetable glycerine, or combinations thereof, such as water and/or an oil. In some embodiments, the liquid carrier may be a water/oil emulsion or an oil/water emulsion.

The liquid carrier may be in an amount between about 70% and about 99% wt % based on total weight of the formulation. The aqueous liquid carrier can be provided in an amount (as a weight % based on total weight of the formulation) of less than about 99, 98, 95, 90, 85, 80, 75 or 70. The aqueous liquid carrier can be provided in an amount (as a weight % based on total weight of the formulation) of at least about 70, 75, 80, 85, 90, 95, 98, or 99. The aqueous liquid carrier can be in a range provided by any two of these upper and/or lower amounts.

The formulation may, for example, comprise a solid carrier. The solid carrier may be selected from the group consisting of plant-based carriers, inorganic carrier, organic carrier, or combinations thereof. For example, the solid carrier may be selected for the group comprising sodium saccharine, sodium bicarbonate, cellulose, magnesium carbonate, magnesium stearate, magnesium silicate (talc), sugar, lactose, mannitol, pectin, dextrin, starch, gelatin. silica, citric acid, maltodextrin, acacia, medium-chain triglyceride (MCT) powder, soy powder, pea protein, and combinations thereof.

In some embodiments, the antimicrobial and/or anti-inflammatory composition comprising an extract of Arthrospira, and at least one organic acid may be absorbed, adsorbed, or retained by the solid carrier.

The solid carrier may be in an amount between about 70% and about 99% wt % based on total weight of the formulation. The solid carrier can be provided in an amount (as a weight % based on total weight of the formulation) of less than about 99, 98, 95, 90, 85, 80, 75 or 70. The solid carrier can be provided in an amount (as a weight % based on total weight of the formulation) of at least about 70, 75, 80, 85, 90, 95, 98, or 99. The solid carrier can be in a range provided by any two of these upper and/or lower amounts.

The solid carrier may be suitably one or more substances which may also act as diluents, flavorant, lubricants, suspending agents, binders or disintegrant.

### Optional Additives

The formulation may further include one or more additives. In some embodiments, the one or more additional additives may be selected from a stabiliser, diluent, adjuvant, dispersing agent, suspending agent, acidifying agent, adsorbent, alkalizing agent, anti-adherent, antioxidant, binder, buffering agent, colorant, complexing agent, filler, direct compression excipient, disintegrant, flavorant, fragrance, glidant, lubricant, opaquant, plasticizer, preservative, or sweetening agent. For example, preservatives, emulsifiers, dispersing agent, and fragrances.

### Methods of Preparing Formulations

The formulations may conveniently be presented in unit dosage form and may be prepared by any of the methods well known in the art. All methods include the step of bringing a composition comprising the antimicrobial and/or anti-inflammatory composition comprising an extract of Arthrospira, and at least one organic acid, as described herein, into association with a carrier, and one or more optional additives.

The plurality of phases includes a fourth phase of formulating the antimicrobial and/or anti-inflammatory composition.

In general, the formulations are prepared by bringing a formulation comprising an antimicrobial and/or anti-inflammatory composition comprising an extract of Arthrospira, and at least one organic acid, as described herein, into association with a liquid carrier to form an emulsion, cream or lotion. Alternatively, the formulations are prepared by bringing a formulation comprising an antimicrobial and/or anti-inflammatory composition comprising an extract of Arthrospira, and at least one organic acid, as described herein, into association with a solid carrier to form a powder formulation.

The plurality of phases includes a fifth phase of testing the antimicrobial and/or anti-inflammatory composition.

### Methods and Uses

The present disclosure provides for a method of treating or preventing a disease or condition responsive to treatment with an Arthrospira extract (e.g. biosource) in a subject, comprising administering to a subject an effective amount of the composition as described herein or the composition prepared by the process as described herein.

The present disclosure also provides for a use of an extract of Arthrospira (e.g biosource) in the manufacture of a medicament for treating or preventing a disease or condition responsive to treatment with an Arthrospira extract, wherein the medicament comprises the composition as described herein or the composition prepared by the process as described herein.

In an embodiment, the antimicrobial and/or anti-inflammatory composition has antimicrobial (for example, chitinase activity) properties, anti-inflammatory properties, and/or cell growth-promoting properties.

In an embodiment, the antimicrobial and/or anti-inflammatory composition is used for preventing or treating onychomycosis or topical microbial/fungal infections.

In an embodiment, the antimicrobial and/or anti-inflammatory composition is used for preventing or treating dermatological conditions such as acne, atopic dermatitis, rosacea, psoriasis, actinic keratosis and skin cancers (such as melanoma and basal cell carcinoma).

In an embodiment, the antimicrobial and/or anti-inflammatory composition reflects an anti-microbial (for example chitinase activity) property, anti-inflammatory property, and cell growth-promoting property. In an embodiment, the antimicrobial and/or anti-inflammatory composition is used for preventing or treating onychomycosis or topical microbial/fungal infections. In an embodiment, the antimicrobial and/or anti-inflammatory composition is used for preventing or treating dermatological conditions such as acne, atopic dermatitis, psoriasis, rosacea, actinic keratosis and skin cancers (e.g melanoma and basal cell carcinoma). In an embodiment, the antimicrobial and/or anti-inflammatory composition is used in the prevention and treatment of pathogenic processes in human beings and animals by any administration method such as oral, topical, or parenteral. In certain embodiments, during the production of the antimicrobial and/or anti-inflammatory composition, it is noted that residual benzoic acid can be produced from the reaction of the reagents used in the production process, in particular during the step of decolorizing and/or deodorizing the “biosource”.

A composition, as described herein, may be used in treating or preventing a disease or condition responsive to treatment with an Arthrospira extract, wherein the composition is as described herein or the composition prepared by the process as described herein.

In any of these embodiments, the disease or condition may be onychomycosis, a fungal infection, a bacterial infection, a viral infection, inflammation, acne, rosacea, a dermatological disorder, hidradentitis, psoriasis, actinic keratosis, atopic dermatitis, pruritis, radiation-induced dermatitis, chemotherapy-induced dermatitis, paronychia or alopecia.

The composition described herein may formulated in an array of different forms. For example, the composition may be formulated in formulations including those suitable by topical administration. By way of example only, the formulation may be in the form of a powder for preparing creams or lotions for application to the skin, or any other form that is effective and safe for administration. In another example, the formulation may be in the form of a powder that is effective and safe for administration.

In some embodiments, the formulation may be powder formulation. The formulation may also be in the form of a tablet or a capsule.

In some embodiments, the formulation may be a topical formulation, such as a cream or lotion.

In some embodiments, the composition is formulated for administration to a mammal by topical administration (e.g. for application to the skin).

In some embodiments, the formulation is in the form of a powder, a tablet, a capsule, an emulsion, a cream, or a lotion. In one example, the formulation is in the form of a powder. Powders may be used in a solid dosage form suitable for topical administration. In some embodiments, solid dosage form preparations, such as powders, may be converted, shortly before use, to liquid dosage form preparations for topical administration, for example. These liquid dosage forms may include dissolved solutions, suspensions, emulsions, creams or lotions.

In some embodiments, liquid dosage form preparations may include solutions, suspensions, and emulsions. It will be appreciated that aqueous solutions, suspensions, and emulsions, suitable for oral, topical or parenteral use, are prepared by dissolving or dispersing the composition in water or other suitable liquid (such as a liquid carrier) and adding suitable additives, as described herein.

The antimicrobial and/or anti-inflammatory formulation can be used in the prevention and/or treatment of pathogenic processes in human beings and animals by any suitable administration method, such as oral, topical, or parenteral.

In a preferred embodiment, the antimicrobial and/or anti-inflammatory formulation is applied topically to skin or nails.

### Dosage Regimen

As used herein, “therapeutically effective amount” refers to a composition as described herein, being applied in an amount sufficient to alleviate or prevent to some extent one or more of the symptoms of the disorder or condition being treated, typically without undue adverse side effects or to achieve a desired pharmacological effect or therapeutic improvement with a reduced side effect profile. The results can be the reduction and/or alleviation of the signs, symptoms, or causes of a disease or condition, or any other desired alteration of a biological system. In some embodiments, the term “therapeutically effective amount” refers to a formulation as described herein, being applied in an amount sufficient to result in a reduction of symptoms associated with, for example, onychomycosis, a fungal infection, a bacterial infection, a viral infection, inflammation, acne, rosacea, a dermatological disorder, hidradentitis, psoriasis, actinic keratosis, atopic dermatitis, pruritis, radiation-induced dermatitis, chemotherapy-induced dermatitis, paronychia or alopecia. Therapeutically effective amounts may, for example, be determined by routine experimentation, including but not limited to a dose escalation clinical trial. The phrase “therapeutically effective amount” includes, for example, a prophylactically effective amount. In some embodiments, a prophylactically effective amount is an amount sufficient to prevent onychomycosis, a fungal infection, a bacterial infection, a viral infection, inflammation, acne, rosacea, a dermatological disorder, hidradentitis, psoriasis, actinic keratosis, atopic dermatitis, pruritis, radiation-induced dermatitis, chemotherapy-induced dermatitis, paronychia or alopecia. It is understood that “an effective amount” or “a therapeutically effective amount” can vary from subject to subject, due to variation of age, weight, general condition of the subject, the condition being treated, the severity of the condition being treated, and the judgment of the prescribing physician. An appropriate “effective amount” or “a therapeutically effective amount” in any individual case may be determined by one of ordinary skill in the art using routine experimentation.

The amount of a composition as described herein, that will be effective in the treatment and/or prevention of a particular disease or condition disclosed herein will depend on the nature of the disease or condition, and can be determined by standard clinical techniques. In addition, in vitro or in vivo assays may optionally be employed to help identify optimal dosage ranges. Such techniques are known to the person skilled in the art.

The precise dose to be administered to the subject will also depend on the route of administration, and the seriousness of the disease or condition, and should be decided according to the judgment of the practitioner and each subject's circumstances.

In some embodiments, a therapeutically effective amount of a composition as described herein, is administered to the subject at a predetermined frequency. In some embodiments, a composition as described herein, is administered to the subject according to a dosage regimen in which a composition as described herein is administered once daily, twice daily, three times daily, or four times daily. In some embodiments, the composition as described herein is administered to the subject according to a dosage regimen in which a composition as described herein is administered once daily. In some embodiments, a composition as described herein is administered to the subject according to a dosage regimen in which a composition as described herein is administered twice daily. In some embodiments, a composition as described herein is administered to the subject according to a dosage regimen in which a composition as described herein is administered three times daily. In some embodiments, a composition as described herein is administered to the subject according to a dosage regimen in which a composition as described herein is administered four times daily.

In some embodiments, a composition as described herein is administered to the subject according to a dosage regimen in which a composition as described herein is administered multiple times daily.

In some embodiments, a therapeutically effective amount of a composition as described herein, is administered to the subject at a predetermined frequency and/or duration. For example, administration according to any embodiments (e.g. frequency) as described herein may be for a duration of about, or at least about, 1 day, 1 week, 2 weeks, 3 weeks, 1 month, 2 months, 3 months, 4 months, 5 months, 6 months, 12 months, 2 years, or 5 years. Administration of the therapeutically effective amount of a composition as described herein, may be ongoing so long as a therapeutic effect is received by the subject.

As used herein, the term “administer” and “administering” are used to mean introducing the composition as described herein, onto the surface of the skin of a subject. When administration is for the purpose of treatment, the composition as described herein, is provided at, or after the onset of, a symptom of a particular disease or condition, such as onychomycosis, a fungal infection, a bacterial infection, a viral infection, inflammation, acne, rosacea, a dermatological disorder, hidradentitis, psoriasis, actinic keratosis, atopic dermatitis, pruritis, radiation-induced dermatitis, chemotherapy-induced dermatitis, paronychia or alopecia. The therapeutic administration of this substance serves to attenuate any symptom, or prevent additional symptoms from arising. When administration is for the purposes of preventing or reducing the likelihood of the disease or condition, e.g. onychomycosis, a fungal infection, a bacterial infection, a viral infection, inflammation, acne, rosacea, a dermatological disorder, hidradentitis, psoriasis, actinic keratosis, atopic dermatitis, pruritis, radiation-induced dermatitis, chemotherapy-induced dermatitis, paronychia or alopecia, the composition as described herein, is provided in advance of any visible or detectable symptom. The prophylactic administration of the composition as described herein, serves to attenuate subsequently arising symptoms or prevent or reduce the likelihood of the symptoms from arising altogether.

The composition may also be used to treat and/or prevent other diseases or conditions, and may include, but not limited to, tinea (e.g. athlete's foot, jock itch, ringworm, deep nail bed infection) which is caused chiefly by species of Microsporum, Trichophyton, Candida and Epidermophyton; thrush, which is caused by Candida; dandruff, which is caused by Malassezia; tropical mycetoma—a granular spreading growth in the skin, muscle tissue and lymph nodes—which is caused by Madurella; sporothricosis—a granular spreading growth in skin and lymph nodes—which is caused by Sporothrix schenkii; and histoplasmosis—a chronic pneumonia with spreading systemic infection—which is caused by Histoplasma capsulatum. The composition can be used to treat, for example, ring worm, hoof infections (Candida, Malassezia), dermatitis and folliculitis (Microsporum, Trichophyton, Alternaria, Fusarium). The composition can be used to treat bacterial infections and infestations. The bacteria may be Gram-positive bacteria and may be, for example, Bacilli, Clostridia, Staphylococci or Pneumococci. The bacteria is preferably Propionibacterium acne, the causative agent of acne. The composition can be used to treat acne, dermatitis, ulcers or wounds caused by or infected with Gram-positive bacteria. The composition can be used to treat and/or prevent skin defects and other dermatological conditions, such as, for example, a pit, acne damage, rosacea, a reddened area, a crack, a burn, a blister, psoriasis, eczema, scaling, wrinkles, a papule, a stomatitis, a lesion, a pustule, a wound, cradle cap, diaper rash, an ulcer, a cold sore, shaving rash, chicken pox, dermatitis, cracked heels and elbows. The composition can be used to treat burns, insect and animal bites, and to remove inflammation of the skin. The composition can be used to reduce itchiness of the skin. The composition can be used to repair scar tissue, sun-damaged skin, and dry and scaly skin that has lost its elasticity.

### EXAMPLES

The present disclosure is further described by the following examples. It is to be understood that the following description is for the purpose of describing particular examples only and is not intended to be limiting with respect to the above description.

### Example 1a—Preparation of Extract of Arthrospira

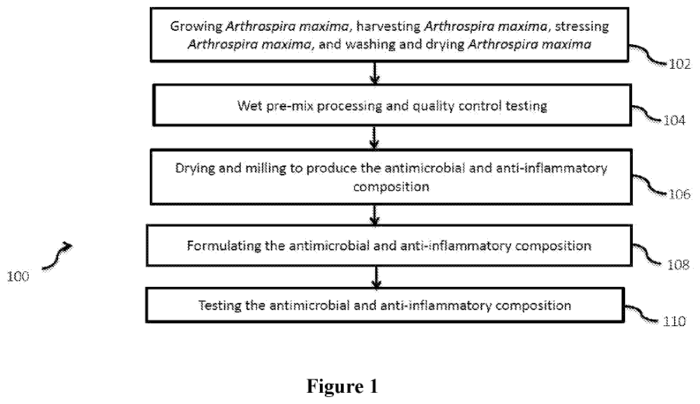

FIG. 1 illustrates a flowchart of the method 100 of producing an antimicrobial and/or anti-inflammatory composition, in accordance with an alternative embodiment of the present invention. The antimicrobial and/or anti-inflammatory composition comprises an extract of Arthrospira, and at least one organic acid.

In the first phase 102, the cyanobacterium Arthrospira maxima is grown in aquaculture ponds in the Northern Territory, Australia and/or Chile. For example, either pure spring water or mineral water is used for growing, and herbicides or pesticides are not used during the growing step. In the first phase 102, the A. maxima is removed from the aquaculture ponds and about 80% water is removed from the culture mass by filtration, and A. maxima is physiologically stressed. The stressed A. maxima is washed and dried in a drying tower to a moisture level of approximately 4-5%. The washed and dried A. maxima provides a fine, and uniform dark green powder (“biosource” or “Arthrospira maxima powder” or “extract of Arthrospira”).

In a preferred embodiment, the biosource is produced as a food substance in accordance with Australian regulatory standards. It is particularly preferred that in-process controls and monitoring are in accordance with GMP procedures throughout the production process.

Following the first phase 102, quality control testing of the Arthrospira maxima powder (biosource or extract of Arthrospira) is then performed. Preferably, the biosource is checked for confirmation that the macroscopic and microscopic morphology is consistent with A. maxima. In addition, it is preferable that microbial load tests, HPLC identification of the biosource as Arthrospira maxima; and fungicidal efficacy testing is undertaken. The biosource is then released for use in the second phase 104 to provide a “wet pre-mix intermediate”.

The second phase 104 preferably takes place as a GMP process in a 1,000-litre tank incorporating an in-line emulsifier and temperature control. During this second phase 104, colour changes and physical characteristics are monitored. The second phase 104 results in the release of the bioactive, the breakdown of the chlorophyll (green colour) and removal of the characteristic biosource odour to produce a pale cream/manila coloured liquid with a mild odour. This pale cream/manila coloured liquid with a mild odour is the “wet pre-mix intermediate”.

### Example 1b—Preparation of “Wet Pre-Mix Intermediate”

Washed and dried A. maxima as a fine, and uniform dark green powder (“biosource” or “Arthrospira maxima powder” or “extract of Arthrospira”) (60 kg), is pre-moistened with water (up to 30 L) in a stainless steel mixing vessel. Typically, this is achieved by gradually adding the water to the powder with constant stirring until a moist, friable, uniform crumble forms. The crumble is then immediately transferred to a suitable vessel for production of a “wet pre-mix intermediate”.

Typically, a suitable vessel is a large tank to which purified water (400 L) has been added. Pre-moistened biosource (Arthospira extract) is added to the water in the vessel, and mixed thoroughly.

An initial quantity of a first agent to decolorize and/or deodorize the Arthrospira extract (e.g. benzoyl peroxide (3.5 kg)) is added to the mixture in the vessel, and mixed in. Addition of the agent, e.g. benzoyl peroxide, results in a change in colour of the mixture from a dark green to an olive hue.

An initial quantity of a second agent to decolorize and/or deodorize the Arthrospira extract (e.g. 50% hydrogen peroxide (6 L)) is added to the mixture and mixed in. A second quantity of the second agent, e.g. 50% hydrogen peroxide (5 L), is then added and mixed in. Following continued mixing, there is a colour change from the olive hue to mocha.

A second quantity of pre-moistened Arthrospira extract (e.g. biosource (30 kg)) is added to the vessel and mixed thoroughly. A second quantity of the first agent, benzoyl peroxide (1 kg), is then added and mixed, resulting in the mixture revering to an olive hue in colour. A third and final quantity of the second agent, 50% hydrogen peroxide, is added and mixed in, resulting in a colour change to orange/mustard.

Titanium dioxide (6 kg) is slowly added and mixed in until the “wet pre-mix intermediate” is a yellow/manila colour.

The “wet pre-mix intermediate” is subjected to microbial load tests, chemical analysis of residuals and fungicidal efficacy testing.

Following satisfactory microbial test results, the wet pre-mix intermediate is optionally subjected to the third phase 106 of drying and milling to produce the antimicrobial and/or anti-inflammatory composition. Drying involves the removal of water molecules from the “wet pre-mix intermediate” by a refractory drying process (typically conducted at ˜25° C.) to give a product with a moisture level of 3%. Temperature and pH are monitored during drying, whilst the moisture content is determined during and after drying. In addition, the colour and appearance of the product are evaluated during and after drying.

Milling of the dried material is preferably a two-stage process to result in a powder with a particle size of ˜60 μm. The resultant powder, which is the antimicrobial and/or anti-inflammatory composition is bagged and vacuum-sealed, preferably under nitrogen flush.

Alternatively, following satisfactory microbial test results, the wet pre-mix intermediate is subjected to a minimal drying phase to give a product with a moisture level of 90%.

In a further alternative, following satisfactory microbial test results, the wet pe-mix intermediate is formulated directly into an antimicrobial and/or anti-inflammatory composition.

Further, the present specification describes the quality control testing of the antimicrobial and/or anti-inflammatory composition and use in the manufacture of finished (formulated) products after receipt of satisfactory quality control testing results. The quality control tests include microbial load tests (including confirming the absence of specific pathogens). The chemical analysis of residuals content is also performed. Fungicidal efficacy tests using simulated formulated products prepared in the laboratory are also undertaken, as is organoleptic assessment (colour, smell).

The plurality of phases includes a fourth phase 108 of formulating the antimicrobial and/or anti-inflammatory composition. The resultant powder from the optional third phase 106, or the “wet pre-mix intermediate” from the second phase 104, is incorporated into formulations such as aqueous-based creams or lotions. The resultant powder from the optional third phase 106, or the “wet pre-mix intermediate” from the second phase 104, can also be incorporated into formulations by blending with a powder base to produce a range of therapeutic products for the topical treatment of fungal skin or nail infections caused by dermatophytes.

The plurality of phases includes a fifth phase 110 of testing the antimicrobial and/or anti-inflammatory composition. The resultant powder from the third phase 106 is subjected to quality control test results, which can include HPLC identification of A. maxima, microbiological testing in accordance with regulatory requirements, and/or fungicidal efficacy testing. Regulatory requirements might also specify other physical and chemical testing. On-going stability testing as required can also be performed.

The organic acid is produced as a by-product of decolorizing and/or deodorizing the “biosource” during production of the “wet pre-mix intermediate”, and has at least one of anti-fungal activity, antimicrobial activity, and a combination thereof. Preferably, the organic acid is benzoic acid, valproic acid or a combination thereof. The following Table 1 presents data showing that a formulated cream using an antimicrobial and/or anti-inflammatory composition prepared using a process of the present invention and the “wet pre-mix intermediate” have greater activity than 0.4% and 0.8% benzoic acid even though the formulated cream may contain that same amount of benzoic acid.

TABLE 1
Activity of formulation

| | Affected zone (mm) | | | | |
|---|---|---|---|---|---|
| | Clearance zone | | Inhibition zone | | |
| Sample | Data | Mean | Data | Mean | Mean Total |
| Base Cream + | 0, 1, 1 | 0.7 | 0, 0, 0 | 0 | 0.7 |
| 0.4% benzoic | | | | | |
| acid | | | | | |
| Base Cream + | 4, 3, 3 | 3.3 | 1, 2, 2 | 1.7 | 5 |
| 0.8% benzoic | | | | | |
| acid | | | | | |
| AMY1045 | 0, 0, 0 | 0 | 7, 6, 8 | 7 | 7 |
| Arthrospira | | | | | |
| maxima cell | | | | | |
| powder 10% | | | | | |
| suspension in | | | | | |
| water subjected | | | | | |
| to sheer stress | | | | | |
| (in a blender) | | | | | |
| AMY1045 | 0, 0, 0 | 0 | 0, 2, 2 | 1.3 | 1.3 |
| Arthrospira | | | | | |
| maxima cell | | | | | |
| powder 10% | | | | | |
| mixed with | | | | | |
| cream | | | | | |
| AMY1045 | 0, 0, 0 | 0 | 5, 5, 4 | 4.7 | 4.7 |
| Arthrospira | | | | | |
| maxima cell | | | | | |
| powder 10% | | | | | |
| ‘blitzed’ | | | | | |
| (comminuted) | | | | | |
| with a Bar Mix | | | | | |
| in cream | | | | | |
| AMY1045 | 18, 18, 18 | 18 | 2, 2, 3 | 2.3 | 20.3 |
| WPM | | | | | |
| AMY1045 | 6, 8, 6 | 6.7 | 0, 0, 0 | 0 | 6.7 |
| APM 8% | | | | | |
| mixed with | | | | | |
| cream | | | | | |

The organic acid is added to the antimicrobial and/or anti-inflammatory composition during the fourth phase 108 of formulating the antimicrobial and/or anti-inflammatory composition.

### Example 2—Preparation of an Antimicrobial and/or Anti-Inflammatory Composition

Purified water is measured into a production vessel (e.g. a stainless-steel tank with an internal stirrer) and an extract of Arthrospira maxima powder is dissolved by slowly adding and with stirring to avoid dispersion.

The other raw materials listed below in Table 2, such as hydrogen peroxide, benzoyl peroxide and titanium dioxide are weighed into the vessel and slowly added sequentially with constant stirring ensuring that the temperature does not exceed 40° C.

Once all materials are added, mixing is continued for at least 48 hours (longer if necessary) to achieve a satisfactory colour change (i.e. from green to a creamy-yellow/manila colour), forming the “Wet Pre-Mix Intermediate”. The Wet Pre-mix is then decanted into Pallecon/Drums via a diaphragm pump for storage. The Wet pre-mix is dried to form a dried antimicrobial and/or anti-inflammatory composition powder that can be stored and reconstituted to a variety of formulations.

TABLE 2
Raw materials used in the preparation of wet pre-mix intermediate

| Ingredient | Quantity | |
|---|---|---|
| Arthrospira maxima cell powder | ~50 | Kg² |
| DiBenzoyl peroxide (75% waterised | 2.42 Kg | |
| synonym = Benzoyl peroxide) | (approx. 0.75% w/w of total | |
| | production batch) | |
| Hydrogen peroxide (at a concentration | 9.50 L | |
| of 50%) | (approx. 2.53% w/w of total | |
| | production batch) | |
| Titanium dioxide | 3.76 Kg | |
| | (approx. 1% w/w of total | |
| | production batch) | |
| Purified Water | 309 | L |

| | |
|---|---|
| Total weight/volume of mixture: | ~375 Kg (approx. 375 L) |

### Example 3—Analysis of Composition

A sample of the antimicrobial and/or anti-inflammatory composition (5 g) prepared according to a process of the invention was extracted with HPLC grade methanol (50 mL) at room temperature over a period of 8 days. The mixture was then filtered through a glass fibre filter paper and the filtrate was concentrated in vacuo on a rotary evaporator at 40° C. The resultant yellow residue (720 mg) was suspended in HPLC grade methanol (7 mL), sonicated for several minutes, then filtered through a 0.45 mm syringe filter to give a final filtrate for analysis.

Aliquots (400 μL) of the final filtrate were separated by reverse-phase HPLC (Waters 600 multisolvent delivery system, with a Waters 486 tunable absorbance detector and a Waters 600E system controller) using a Phenomix Betasil-C-18 Thermo-Hypersil-Keystone semi-preparative column (150 mm×10 mm, 5 mm) connected to a Alltech Platinum EPS C18 preparative column (150 mm×22 mm, 5 mm). The column separation was performed as a gradient elution method using an aqueous 0.025M KH₂PO₄/methanol mixture at a flow rate of 5 mL/min using a wavelength of 230 nm for detection. The gradient elution program is set out in Table 3.

TABLE 3
Gradient elution program

| Time (min) | Methanol (%) | Aqueous KH₂PO₄ (%) |
|---|---|---|
| 0 | 30 | 70 |
| 9 | 35 | 65 |
| 15 | 100 | 0 |
| 20 | 100 | 0 |
| 23 | 30 | 70 |
| 30 | 30 | 70 |

Three fractions were collected with retention times ranging from 0 to 12 minutes, (Fraction 1), 12 to 16 minutes (Fraction 2) and greater than 16 minutes (Fraction 3). Fractions 1 and 3 were discarded as having no antifungal activity, and Fraction 2 was retained for further analysis.

Fraction 2 was evaporated to give a colourless solid (1.02 g). Extraction into methanol (3×7 mL) and subsequent evaporation of the solvent afforded a pale yellow solid (98 mg).

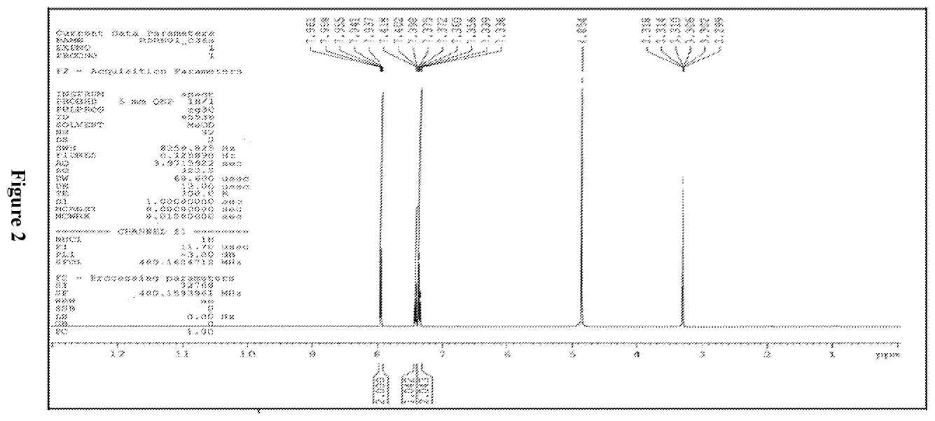

The pale yellow solid (5 mg) was dissolved in d4-methanol and analysed by proton nuclear magnetic resonance (′H NMR) (Bruker 400 MHz). The spectrum is shown in FIG. 2, and has six distinct signals at δ 3.31 ppm (residual solvent), δ 4.85 ppm (residual water), δ 7.34-7.37 ppm, δ 7.40-7.42 ppm and δ 7.93-7.96 ppm. The signals between δ 7.34 and δ 7.96 ppm are typical of a mono-substituted phenyl system with a doublet at δ 7.96 ppm representing two ortho protons, a triplet at δ 7.40 ppm corresponding to the para proton and a multiplet at δ 7.36 ppm which can be assigned to the meta protons in such an aromatic system.

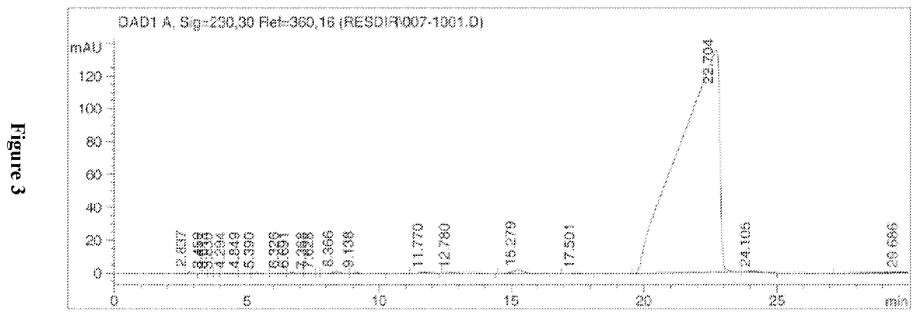

The methanol extract of Fraction 2 was also analysed by reverse phase HPLC using a modular Hewlett Packard HP1100 Chemstation, with a UV-DAD in combination with an Alltech Platinum C-18 column (250 mm×4 mm i.d.×5 mm) and an isocratic elution method using an aqueous KH₂PO₄ (0.025 M)/methanol mixture (95%:5%) at a flow rate of 1 mL/min. The chromatogram is shown in FIG. 3, and has one major peak with a retention time of 22.7 minutes and a purity of 98.1%.

An authentic sample of benzoic acid was analysed under the same HPLC conditions, and had the same retention time as Fraction 2.

The NMR and HPLC results suggest that benzoic acid has been isolated from the antimicrobial and/or anti-inflammatory composition in high purity.

### Example 4—Anti-Inflammatory Activity of Reconstituted Dried Extract—In Vitro Skin Model—Inhibition of Interleukin-1 Alpha

Anti-inflammatory properties of a composition of the invention was tested using three concentrations of the composition prepared, as described in Example 2, and is mixed with distilled water to form an emulsion of 8%, 18% and 30% in a reconstructed artificial human skin model comprising normal human epidermal keratinocytes, growing as an integrated three-dimensional cell culture model (Skinethic, Nice, France), which was utilised as an in vitro mimic of human skin.

The model exhibits normal barrier functions, due to the presence of a differential stratum corneum). Normal human epidermal keratinocytes were seeded on a collagen matrix and grown in a serum-free medium to reach a multilayer conformation with a differentiated stratum corneum at the surface. Epidermis units having a 0.5 cm² diameter were purchased directly from Skinethic at the 16th day of culture. The epidermis layer was placed in a transwell chamber, on a porous membrane. Undiluted duplicate samples of the products to be tested (10-15 mg) were applied on the upper keratinised layer of skin, with or without pre-treatment with 0.25% SLS (30 minutes). After 24 hours at 37° C. under 5% CO², the samples were removed and the skin washed twice with PBS. An MTT assay was then undertaken to evaluate cell survival and therefore to assess irritation potential of the samples. The key component of the MTT assay, is (3-[4,5-dimethylthiazol-2-yl]-2,5-diphenyl tetrazolium bromide (MTT) which is yellow-coloured in solution. Mitochondrial dehydrogenases of viable cells cleave the tetrazolium ring, leading to the formation of purple crystals which are insoluble in aqueous solutions. The crystals are re-dissolved in acidified isopropanol and the resulting purple solution is measured spectrophotometrically. An increase or decrease in cell number results in a concomitant change in the amount of formazan formed, indicating the degree of cytotoxicity caused by the test material.

In the present assays, after exposure of the cells to the test material, the cells were washed with Dulbecco's PBS. After removal of the PBS, the MTT-medium was added to each culture well and the cells were incubated at 37° C. for four hours. Following incubation, the MTT-medium was removed and an MTT solubilisation solution (acidified isopropanol) was added to each well. The culture plate was shaken on a gyratory plate shaker for 20-30 minutes, ensuring that all the crystals had dissolved and formed a homogenous solution. All three concentrations had the same level of cell viability indicating that there were no cytotoxic effects on skin even up to 30% of composition as described herein.

Inhibition of the release of IL-1 alpha was measured as an indicator of skin irritation, using commercially available ELISA test kits. A standard plot designed with the cytokine IL-1 alpha allows the titration in the cell medium by reading the absorbance at defined wavelengths, after specific identification with colorimetric reactions based on antibodies. For this assay, 200 μL of culture medium in the lower compartment of the well was collected at 6 hours and 24 hours. A solution of 0.25% SLS (sodium lauryl sulfate) was used as the positive control while treated samples included the Algal Extract+0.25% SLS. Culture media served as negative control. The results are summarized below in Table 4.

TABLE 4
Inhibition of interleukin-1 alpha

| Composition | IL-1 alpha inhibition | IL-1 alpha inhibition |
|---|---|---|
| Concentration | (after 6 hours) | (after 24 hours) |
| 8% | No inhibition | 16.28% |
| 18% | 5.64% | 39.89% |
| 30% | 22.54% | 58.70% |

The results show that up to 30% of the composition did not show any toxicity in vitro in a reconstructed 3-dimensional human skin model. The anti-inflammatory activity was dose-dependent based on the decreased secretion of the cytokine interleukin-1 alpha with increasing concentrations of the composition when tested in reconstructed 3-dimensional human skin model. Thus, the process has produced a antimicrobial and/or anti-inflammatory composition that has anti-inflammatory properties in spite of increasing amounts of an irritant (benzoic acid) and bleaching of an anti-inflammatory factor, phycocyanin.

### Example 5—Anti-Inflammatory Activity of Reconstituted Dried Extract—Effect on Sebum Secretion in Acne

An 8% formulation of the composition described herein was tested on 20 male and female volunteers with acne, in order to evaluate the tested product efficacy in normalizing the skin sebaceous secretion. These evaluations provide the comparison of sebum level between the area of interest before the beginning of the treatment (TO days) and the sebum level measured after 30 days of treatment (T30 days). Sebum secretion was measured using a Sebumeter (Sebumeter® SM815, Courage-Khazaka GmbH, Germany). The measuring principle of this instrument is based on a photometric method not influenced by the skin moisturization. The probe used for this measurement contains a plastic opaque tape 0.1 mm thick. A little mirror under the measuring portion of the tape protrudes from the measuring top of 1 mm. The mirror is connected to the tape through a spring calibrated at 0.3N. This ensure that the contact of the tape to the skin is maintained with a constant pressure. The probe is applied perpendicularly on the test area and kept in contact with the skin for 30±2 seconds. In order to determine the amount of sebum the probe is inserted into a photometer that measures the light transmission before and after the contact with the skin. The light transmission is related to the amount of sebum on the tape. A microprocessor calculates the result and show it on the display expressed in unit according to a scale from 0 to 350 μg/cm². The results are summarised below in Table 5.

TABLE 5

| | | |
|---|---|---|
| Time Points | Treated | Untreated |

Mean sebum values (μg/cm²)

| | | |
|---|---|---|
| T0 days | 220.45 | 216.95 |
| T30 days | 193.75 | 217.80 |

Percentage variations of the mean sebum values (T30 days vs T0 days)

| | | |
|---|---|---|
| T30 days vs T0 days | −12.29% | 0.44% |

Furthermore, a clinical evaluation was conducted on the subjects participating in the study at the start of the study (TO) and after 30 days (T 30 days) of daily use of the product. At each observational time the application area was carefully examined for the following parameters reported—redness, itching, shiny and oily skin, appearance of pores, smoothness and mattifying effect. Each parameter was scored with values ranging from 0 to 4 to express increasing severity of the observed conditions as shown below in Table 6.

TABLE 6
Clinical evaluation

| Parameters | T0 Days | T30 Days |
|---|---|---|
| Redness | 1.0 | 0.4 |
| Itching | 1.1 | 0.6 |
| Shiny and oily skin | 2.3 | 1.4 |
| Appearance of pores | 2.2 | 1.6 |
| Smoothness | 2.4 | 1.8 |
| Mattifying effects | 3.6 | 1.6 |
| MEAN SCORES | 12.5 | 7.35 |

The results show that application of 8% of the antimicrobial and/or anti-inflammatory composition described here leads to significant reduction of sebum secretion and severity of clinical parameters in acne subjects.

### Example 6—Chemical Analysis of Dried Extract

### Analysis of Flavonoids

Homogenised Arthrospira extract was weighed and extracted with 80% Methanol. Extracts are sonicated, centrifuged and then loaded onto a clear 96-well microplate. Water and 5% (w/v) NaNO₃ are added to each well. The plate is allowed to incubate at room temperature. The FLUOstar OPTIMA microplate reader then injects 10% (w/v) aluminium-chloride solution, incubates for 1 minute at room temperature and then adds 1M NaOH. This results in a spectral shift of all aluminium-complexed flavonoids from all other phenolic compounds. The absorbance of each well is then read at wavelength of 510 nm. Amounts are deduced from a standard curve. (REF: Marinova D, Ribarova F, Atanassova M. Total phenolics and flavonoids in Bulgarian fruits and vegetables. Journal of the University of Chemical Technology and Metallurgy 2005, 40(3):255-260.

### Analysis of Polyphenols

Homogenised Arthrospira extract was weighed and extracted with 80% Methanol. Extracts are sonicated, centrifuged and then loaded onto a clear 96-well microplate. Water is added to each well and the plate is loaded onto the FLUOstar OPTIMA instrument. The FLUOstar OPTIMA microplate reader then injects Folin-Ciocalteu reagent, incubates for 5 minutes at room temperature then adds Na₂CO₃. The plate is incubated at room temperature for a further 90 minutes. The absorbance of each well is then read at wavelength of 750 nm. Amounts are deduced from a standard curve. (REF: Marinova D, Ribarova F, Atanassova M. Total phenolics and flavonoids in Bulgarian fruits and vegetables. Journal of the University of Chemical Technology and Metallurgy 2005, 40(3):255-260.

TABLE 7
Comparison of the composition as described
herein with prior art composition

| | Arthrospira maxima | |
|---|---|---|
| | (Spirulina) dried algae | Dried |
| | (prior art composition) | Composition |
| Flavonoids | 0.13-0.5 g/100 g | 2.45 g/100 g |
| Polyphenols/phenolic acids | 0.45-1.7 g/100 g | 0.62 g/100 g |

### Analysis of Fatty Acids

The dried algal extract (10 g) is homogenised and fat is extracted from the sample using chloroform/methanol. The extract is evaporated under nitrogen. A minimum extracted mass of 0.2 g is required. The extracted fat is esterified using a methanolic sodium methoxide solution and treatment with sulphuric acid in methanol. The solution is neutralised and re-extracted using n-hexane. The hexane layer is removed, dried using anhydrous sodium sulphate and made to volume with hexane. The fatty acids are in the form of their respective methyl ester (Fatty Acid Methyl Esters or FAMEs). The relative proportion of each fatty acid methyl ester in the prepared sample is determined using gas chromatography with flame ioinisation detection. Identification of the individual fatty acids is made by retention time against a standard of known fatty acid methyl esters including both cis and trans isomers. Integration and calculation of proportional fatty acid concentrations is made using instrumental software. The results obtained are proportional only as percentage (or g/100 g) of the FAMEs present in the fat extracted from the sample. Results in Table 8 below.

TABLE 8
Fatty acid composition of dried composition
(only fatty acids >1% are listed)

| | Amount (g/100 g) |
|---|---|
| | or percentage |
| Total fatty acids (saturated and unsaturated) | 4.1 g/100 g |
| Lauric Acid | 3.4% |
| Palmitic acid | 73.5% |
| Stearic acid | 2.2% |
| Oleic acid | 2.1% |
| Linoleic acid | 11.0% |
| Gamma Linolenic acid | 4.6% |
| Docosadienoic acid | 1.8% |

The HPLC analysis of the dried composition prepared, as described in Example 2, shows the presence of 2.6 g/100 g of benzoic acid (2.6%). The analysis reveals that the composition described herein comprises other flavonoids, polyphenols/phenolic acids and fatty acids present in significant quantities when compared with prior art compositions, see for example Table 7 and 8 above.

1. An antimicrobial and/or anti-inflammatory composition comprising an extract of Arthrospira, and at least one organic acid in an amount of at least about 0.1 wt. % based on the total weight of the composition.

2. The composition of claim 1, wherein the organic acid is selected from the group comprising aromatic carboxylic acids, short chain fatty acids, medium and long chain saturated fatty acids, medium and long chain unsaturated fatty acids, polyphenols, phenolic acids, flavonoids, or a combination thereof.

3. The composition of claim 1, wherein the organic acid is selected from the group comprising benzoic acid, valproic acid, formic acid, acetic acid, propionic acid, butyric acid, isobutyric acid, valeric acid, isovaleric acid, cholorogenic acid, gallic acid, vanillin, salicylic acid, caffeic acid, syringic acid, coumaric acid, ferulic acid, cinnamic acid, quercetin, genstein, kaempferol, lauric acid, palmitic acid, sapienic acid, stearic acid, oleic acid, linoleic acid, gamma linolenic acid, docosodienoic acid or a combination thereof.

4. The composition of claim 1, wherein the at least one organic acid is benzoic acid.

5. The composition of claim 4, wherein the amount of benzoic acid is between about 2 wt. % and about 15 wt. % based on the total weight of the composition.

6. The composition of claim 1, wherein the composition further includes titanium dioxide.

7. The composition of claim 6, wherein the amount of titanium dioxide is between about 0.5 wt. % and about 3.0 wt. % based on the total weight of the composition.

8. The composition of claim 1, wherein the moisture content of the composition is less than about 20 wt. %.

9. The composition of claim 1, wherein the composition is formulated for administration to a mammal by topical administration.

10. The composition of claim 1, wherein the composition is in the form of a powder, cream or lotion.

11. A process for preparing an antimicrobial and/or anti-inflammatory composition comprising an extract of Arthrospira, and at least one organic acid, comprising the following steps:
(i) obtaining an extract of Arthrospira; and
(ii) adding at least one agent to decolorize and/or deodorize the Arthrospira extract to provide the antimicrobial and/or anti-inflammatory composition, wherein the addition of the at least one agent produces the organic acid.

12. The process of claim 11, wherein the agent to decolorize and/or deodorize is selected from the group comprising hydrogen peroxide, benzoyl peroxide, valproate peroxide, acetyl peroxide, formyl hydroperoxide, peroxypropionic acid, peroxybutyric acid, or a combination thereof.

13. The process of claim 11, wherein the agent is selected from hydrogen peroxide and benzoyl peroxide.

14. The process of claim 13, which further includes adding at least one organic acid.

15. A process for preparing an antimicrobial and/or anti-inflammatory composition comprising an extract of Arthrospira, and at least one organic acid, comprising the following steps:
(i) obtaining an extract of Arthrospira; and
(ii) adding at least one organic acid to the Arthrospira extract to provide the antimicrobial and/or anti-inflammatory composition.

16. The process of claim 15 which further includes growing and harvesting Arthrospira.

17. The process of claim 15, wherein the Arthrospira were/are grown under physiologically stressed conditions.

18. The process of claim 15 which further comprises rinsing and/or drying the Arthrospira extract to provide a dried Arthrospira powder.

19. The process of claim 15 which further includes the addition of titanium dioxide.

20. The process of claim 15, further comprising addition of a diluent, wherein the diluent is water.

21-31. (canceled)
